(19) United States
(12) Patent Application Publication
(10) Pub. No.: US 2015/0133324 A1
(43) Pub. Date: May 14, 2015

(54) MULTIPLEX REAL-TIME PCR DETECTION OF INFLUENZA VIRUSES 2009 H1N1, INFLUENZA A AND INFLUENZA B

(71) Applicant: VELA OPERATIONS PTE. LTD., Singapore (SG)

(72) Inventors: Xin Zhang, Singapore (SG); Hongliang Qian, Singapore (SG)

(21) Appl. No.: 14/381,569

(22) PCT Filed: Feb. 28, 2013
(86) PCT No.: PCT/IB2013/051605

§ 371 Date: Aug. 27, 2014

(30) Foreign Application Priority Data
Feb. 29, 2012 (GB) 1203547.3

(51) Int. Cl. C12Q 1/70

(52) U.S. Cl. CPC C12Q 1/701; C12Q 2600/16
USPC 506/9; 506/16

(57) ABSTRACT

The present invention relates to assays, diagnostic kits and methods for the simultaneous real-time PCR detection of influenza viruses selected from influenza A subtypes H1 and/or H3 and/or influenza 2009 H1N1 and/or influenza B.

### TECHNICAL FIELD

The present invention relates to the diagnosis of influenza infections, in particular the detection of sub-types of influenza A, influenza B strains and also the detection of influenza A 2009 H1N1.

### BACKGROUND

Influenza virus is an infectious microorganism belonging to the family of Orthomyxoviridae. There are two main types of influenza (flu) virus: Types A and B. The influenza A and B viruses that routinely spread in people (human influenza viruses) are responsible for seasonal flu epidemics each year. Influenza A viruses can be broken down into sub-types depending on the genes that make up the surface proteins. Over the course of a flu season, different types (A & B) and subtypes (influenza A) of influenza circulate and cause illness. Influenza A viruses are divided into subtypes based on two proteins on the surface of the virus: the hemagglutinin (H) and the neuraminidase (N). There are 16 different hemagglutinin subtypes and 9 different neuraminidase subtypes. Influenza A viruses can be further broken down into different strains. Current subtypes of influenza A viruses found in people are influenza A (H1N1) and influenza A (H3N2) viruses. In the spring of 2009, a new influenza A (H1N1) virus emerged to cause illness in people. This virus was very different from regular human influenza A (H1N1) viruses and the new virus caused the first influenza pandemic in more than 40 years. That virus (often called “2009 H1N1”) has now mostly replaced the H1N1 virus that was previously circulating in humans. Influenza B viruses are not divided into subtypes, but can be further broken down into different strains. (cf. www.cdc.gov/flu).

Influenza viruses are enveloped RNA viruses and are capable of infecting the respiratory tract of birds and mammals. Influenza A is the most virulent human influenza pathogen and causes the most severe disease. According to the WHO (Influenza (Seasonal), World Health Organization, April 2009), influenza spreads around the world in seasonal epidemics and results in the deaths of about 250,000 to 500,000 people a year. In pandemic years, this number may rise to millions.

Influenza vaccines are available, but vaccination must be refreshed every year owing to the high mutation rate of the viral RNA resulting in different viral strains predominating each new flu season. Numbers of vaccinated individuals in the total population are frequently not sufficient to prevent epidemics or even pandemics. One of the reasons is that the production of vaccine is currently performed in embryonated eggs, which is time-consuming and may not yield sufficient quantities of vaccine for large epidemics or pandemics, in particular when new virus strain arise during the flu season.

Accordingly, rapid and specific diagnosis of influenza virus nucleic acids is crucial for initial detection, successful outbreak control within hospitals and the community, isolation of patients from others and for directing treatment.

The main object of the present invention is to provide assays, kits, compositions and methods suitable for the detection and/or diagnosis of influenza virus nucleic acids in biological samples, wherein the method is simple, highly specific and suitable for simultaneously detecting the presence or absence of different influenza virus subtypes and strains, respectively.

Accordingly, the present invention relates to a set of nucleic acids, useful for simultaneous detection of influenza 2009 H1N1 virus, influenza A virus and influenza B virus in a biological sample, the set comprising (i) a first pair of primers and a first probe, specific for human influenza 2009 H1N1 virus; (ii) a second pair of primers and a second probe, specific for human influenza A; and (iii) a third pair of primers and a third probe, specific for human influenza B.

In a first embodiment, the set of primers suitable for amplification of 2009 H1N1 influenza virus comprises oligonucleotides selected from SEQ ID Nos: 1 to 3 or oligonucleotides that are at least 80%, 85%, 90% or 95% homologous or identical, or complements of SEQ ID Nos: 1 to 3, and preferably the first probe has a sequence as shown in SEQ ID No: 4, or a complementary sequence or is an at least 80%, 85%, 90% or 95% homologous or identical derivative thereof.

In a second embodiment, the set of primers suitable for amplification of influenza A virus comprises oligonucleotides selected from SEQ ID Nos: 5 to 10 or oligonucleotides that are at least 80%, 85%, 90% or 95% homologous or identical thereto, or complements of SEQ ID Nos: 5 to 10, and preferably the second probe has a sequence as shown in SEQ ID No: 11 or 12, or complementary sequences or at least 80%, 85%, 90% or 95% homologous or identical derivatives thereof.

In a third embodiment, the set of primers suitable for amplification of influenza B virus comprises oligonucleotides selected from SEQ ID Nos: 13 to 17 or oligonucleotides that are at least 80%, 85%, 90% or 95% homologous or identical, or complements of SEQ ID Nos: 13 to 17, and preferably the third probe has a sequence as shown in SEQ ID No: 18, or a complementary sequence or is an at least 80%, 85%, 90% or 95% homologous or identical derivative thereof.

The present invention also relates to a method for the simultaneous detection of influenza A virus, influenza B virus and 2009 H1N1 influenza virus in a biological sample from a patient, comprising:
a) providing a biological sample from a patient;
b) extracting viral RNA from the biological sample;
c) carrying out a RT-PCR with the set of nucleic acids according to the present invention; and
d) detecting amplification products for each virus, wherein the presence of an amplification product is indicative of the presence of the respective virus in the biological sample.

In an alternative method, instead of carrying a RT-PCR, the method comprises a step of reverse-transcription and a step of PCR amplification.

The present invention further concerns the use of a set of nucleic acids according to the present invention for simultaneously detecting 2009 H1N1 influenza and/or influenza A and/or influenza B. It further concerns a method of simultaneously detecting 2009 H1N1 influenza and/or influenza A and/or influenza B by using a set of oligonucleotide primers and probes according to the present invention.

In addition, it concerns a set of oligonucleotide primers and probes according to the present invention for preparing a diagnostic kit useful for simultaneously detecting 2009 H1N1 influenza and/or influenza A and/or influenza B. Optionally, the kit further comprises other components such as a DNA polymerase, a reverse-transcriptase, RNase inhibitors, dNTPs and a PCR and/or RT-buffers.

### SHORT SUMMARY OF PREFERRED EMBODIMENTS

Some of the preferred embodiments of the invention are depicted below:
i. A method for the simultaneous detection of the presence or absence of at least one nucleic acid of 2009 H1N1 influenza and/or influenza A and/or influenza B in a biological sample, wherein the method comprises conducting real-time PCR.
ii. The method according to (i) further comprising isolating nucleic acids from the biological sample and performing a reverse transcription step.
iii. The method according to any one of (i) or (ii), wherein the at least one nucleic acid of 2009 H1N1 influenza and/or influenza A and/or influenza B in a biological sample, wherein primer sets that are specific for 2009 H1N1 influenza and/or influenza A and/or influenza B are used.
iv. The method according to any one of (i) to (iii), wherein the 2009 H1N1 influenza-specific primer set comprises oligonucleotides selected from sequences set forth in SEQ ID Nos: 1 to 3, or complements or sequences having at least about 80%, 85%, 90% or 95% homology or identity.
v. The method according to any one of (i) to (iii), wherein the influenza A-specific primer set comprises oligonucleotides selected from sequences set forth in SEQ ID NO: 5 to 10 and 19 to 22, or complements or sequences having at least about 80%, 85%, 90% or 95% homology or identity.
vi. The method according to any one of (i) to (iii), wherein the influenza B-specific primer set comprises oligonucleotides selected from sequences set forth in SEQ ID NO: 12 to 16 and 23 to 26, or complements or sequences having at least about 80%, 85%, 90% or 95% homology or identity.
vii. The method according to any one of (i) to (vi), wherein at least one probe specifically binding to a nucleic acid of 2009 H1N1 influenza and/or influenza A and/or influenza B is used.
viii. The method according to any one of (i) to (vii), wherein the at least one probe is selected from the oligonucleotides set forth in SEQ ID Nos: 4, 11, 12 and 18.
ix. The method according to any one of (i) to (viii), wherein the primers and or probes carry a fluorescent moiety.
x. A method for the diagnosis of an influenza virus infection comprising performing one of the methods according to any one of the preceding embodiments.
xi. A method for monitoring the treatment of influenza virus infection, said method comprising performing the method according to embodiment (x) before treatment with at least one anti-viral drug and during and/or after treatment with said anti-viral drug.
xii. An assay for simultaneous detection of at least nucleic acid of 2009 H1N1 influenza and/or influenza A and/or influenza B in a biological sample comprising primers specifically hybridizing to nucleic acids derived from said 2009 H1N1 influenza and/or influenza A and/or influenza B, wherein said assay is suitable for real-time PCR.
xiii. The assay according to (xii), wherein the assay comprises primers and/or probes set forth in any one of the preceding embodiments.
xiv. The assay according to embodiment (xii) or (xiii), wherein the assay is adapted for use in a fully automated laboratory.
xv. A diagnostic composition comprising primers and/or probes set forth in any one of the preceding embodiments.
xvi. A diagnostic kit for the simultaneous detection of 2009 H1N1 influenza and/or influenza A and/or influenza B in a biological sample comprising primers and/or probes set forth in any one of the preceding embodiments and optionally comprising instructions for use.
xvii. The diagnostic kit according to embodiment (xvi), wherein said kit further comprises enzymes, deoxynucleotides, and/or buffers for performing a reverse transcription step and/or a PCR step.
xviii. The diagnostic kit according to any one of embodiments (xvi) or (xvii) further comprising reagents for the isolation of nucleic acids from a biological sample.

### SUMMARY

The invention provides for methods of identifying RNA of 2009 H1N1 influenza and/or influenza A and/or influenza B viruses by real-time polymerase chain reaction (PCR) in a biological sample.

Primers and probes for detecting 2009 H1N1 influenza and/or influenza A and/or influenza B are also provided by the invention, as are kits or compositions containing such primers and probes.

Methods of the invention can be used to identify RNA from specimens for diagnosis of 2009 H1N1 influenza and/or influenza A and/or influenza B infection. The specific primers and probes of the invention that are used in these methods allow for the amplification and monitoring the development of specific amplification products.

In particular a multi-plex assay for 2009 H1N1 influenza and/or influenza A and/or influenza B is provided, which allows for simultaneous detection and/or diagnosis of large numbers of different virus subtypes and strains, respectively.

According to one aspect of the invention, a method for detecting the presence or absence of 2009 H1N1 influenza and/or influenza A and/or influenza B in a biological sample from an individual is provided. As influenza viruses are RNA viruses, the method comprises a reverse transcription step, at least one cycling step, which includes an amplifying step and a hybridizing step. The amplifying step includes contacting the sample with at least one pair of specific primers to produce an amplification product if a target influenza nucleic acid molecule is present in the sample. The hybridization step includes contacting the sample with specific probes. In the multiplex assays of the present invention several primer pairs are used that are suitable to hybridize to nucleic acids of specific virus subtypes and strains, respectively, but not to other nucleic acids of other subtypes and strains. As a result of the methods described herein, the simultaneous amplification and subsequent detection of the target subtypes and strains is possible. A pair of influenza primers comprises a first influenza primer and a second influenza primer. Sequences of the primers and the probes of the invention are shown in the sequence listing. It is understood that the method, kits, compositions and product can involve all of the specific primers as long as they are suitable to generate a specific amplification product. Furthermore, all of the specific probes may be used and comprised in the methods, kits, etc. of the invention.

In some aspects of the invention, the primers and/or probes of the invention can be labeled with a fluorescent moiety. Fluorescent moieties for use in real-time PCR detection are known to persons skilled in the art and are available from various commercial sources, e.g. from Life Technologies™ or other suppliers of ingredients for real-time PCR.

Representative biological samples from the respiratory tract include throat swabs, throat washings, nasal swabs, and specimens from the lower respiratory tract. In addition, the cycling step can be performed on a control sample. A control sample can include the same portion of the influenza nucleic acid molecule. Alternatively, a control sample can include a nucleic acid molecule other than an influenza nucleic acid molecule.

Cycling steps can be performed on such a control sample using a pair of control primers and a pair of control probes. The control primers and probes are different from influenza primers and probes. One or more amplifying steps produces a control amplification product. Each of the control probes hybridizes to the control amplification product.

In another aspect of the invention, there are provided articles of manufacture, or kits. Kits of the invention can include at least one pair of specific primers for the amplification of 2009 H1N1 influenza and/or influenza A and/or influenza B and at least one influenza probe hybridizing specifically with the amplification products. Articles of manufacture can include fluorophoric moieties for labeling the primers or probes or the primers and probes are already labeled with donor and corresponding acceptor fluorescent moieties. The article of manufacture can also include a package insert having instructions thereon for using the primers, probes, and fluorophoric moieties to detect the presence or absence of 2009 H1N1 influenza and/or influenza A and/or influenza B in a sample.

In another aspect of the invention, there is provided a method for detecting the presence or absence of 2009 H1N1 influenza and/or influenza A and/or influenza B in a biological sample from an individual. Such a method includes performing at least one cycling step. A cycling step include at least one amplifying step and a hybridizing step. Generally, an amplifying step includes contacting the sample with a pair of primers to produce an amplification product if an influenza nucleic acid molecule is present in the sample. Generally, a hybridizing step includes contacting the sample with an influenza-specific probe. The probe is usually labeled with at least one fluorescent moiety. The presence or absence of fluorescence is indicative of the presence or absence of 2009 H1N1 influenza and/or influenza A and/or influenza B in said sample. Amplification generally involve the use of a polymerase enzyme. Suitable enzymes are known in the art, e.g. Taq Polymerase, etc.

In another aspect of the invention, there is provided a method for detecting the presence or absence of 2009 H1N1 influenza and/or influenza A and/or influenza B in a biological sample from an individual. Such a method includes performing at least one cycling step. A cycling step can include an amplifying step and a dye-binding step. An amplifying step generally includes contacting the sample with a pair of influenza-specific primers to produce an influenza amplification product if an influenza nucleic acid molecule is present in the sample. A dye-binding step generally includes contacting the influenza amplification product with a double-stranded DNA binding dye. The method further includes detecting the presence or absence of binding of the double-stranded DNA binding dye into the amplification product. According to the invention, the presence of binding is typically indicative of the presence of influenza nucleic acid in the sample, and the absence of binding is typically indicative of the absence of influenza nucleic acid in the sample. Such a method can further include the steps of determining the melting temperature between the amplification product and the double-stranded DNA binding dye. Generally, the melting temperature confirms the presence or absence of 2009 H1N1 influenza and/or influenza A and/or influenza B nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for 2009 H1N1 influenza and/or influenza A and/or influenza B. Treatment for influenza can include, e.g., administration of a neuraminidase inhibitor (e.g., oseltamivir phosphate) to the individual. The invention also provides for the use of the articles of manufacture described herein to determine whether or not an individual is in need of treatment for influenza.

Further, the methods and/or the articles of manufacture described herein can be used to monitor an individual for the effectiveness of a treatment for influenza as well as in epidemiology to monitor the transmission and progression of influenza from individuals to individuals in a population. The methods and/or the articles of manufacture (e.g., kits) disclosed herein can be used to determine whether or not a patient is in need of treatment for influenza.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting 2009 H1N1 influenza and/or influenza A and/or influenza B virus nucleic acids in a biological sample that is more sensitive and specific than existing assays is described herein.

Primers and probes for detecting 2009 H1N1 influenza and/or influenza A and/or influenza B infections and articles of manufacture containing such primers and probes are also provided.

The increased sensitivity of real-time PCR for detection of 2009 H1N1 influenza and/or influenza A and/or influenza B as well as the improved features of real-time PCR including sample containment and real-time detection of the amplified product, make feasible the implementation of this technology for routine diagnosis of influenza infections in the clinical laboratory.

The invention provides methods to detect influenza by amplifying, for example, a portion of an influenza nucleic acid derived from 2009 H1N1 influenza and/or influenza A and/or influenza B. Nucleic acid sequences from influenza A are available, e.g. in the Influenza Sequence Database (ISD) (flu.lanl.gov on the World Wide Web, described in Macken et al., 2001, “The value of a database in surveillance and vaccine selection” in Options for the Control of Influenza IV. A.D.M.E., Osterhaus & Hampson (Eds.), Elsevier Science, Amsterdam, pp. 103-106).

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from 2009 H1N1 influenza and/or influenza A and/or influenza B, respectively.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise. The term “multiplex” refers to multiple assays that are carried out simultaneously, in which detection and analysis steps are generally performed in parallel. As used herein, a multiplex assay may also be an assay that is suitable to simultaneously amplify and identify different target nucleic acids of one particular influenza virus subtype or strain.

Within the context of the present invention, a multiplex assay would be for example, a molecular assay that simultaneously screens for 2009 H1N1 influenza and/or influenza A and/or influenza B.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “homology” means that a sequence, e.g. a primer or probe sequence disclosed herein, is essentially identical to the sequence of said primer or probe, but may instead of deoxyribunucleotides comprise corresponding ribonucleotides or synthetic analogues. Homologs of a given sequence hybridize to the same target sequence and permit amplification of a target region in a gene of interest, or they bind to target regions as probes and may be detected, e.g. because they carry fluorescent moieties. Identical sequences correspond to the sequences of the primers and/or probes of the present invention, but they may not be 100% identical, e.g. when one or more residues of the total sequences have been replaced by another residue, or because the 5′ or 3′ ends of the primers/probes disclosed herein have been shortened or lengthened. Such primers/probes maintain their capability of hybridizing with a target region and permitting amplification or detection of said target region.

As used herein, the term “target amplification” refers to enzyme-mediated procedures that are capable of producing billions of copies of nucleic acid target. Examples of enzyme-mediated target amplification procedures known in the art include PCR.

Within the context of the present invention, the nucleic acid “target” is the nucleic acid sequence of 2009 H1N1 influenza and/or influenza A and/or influenza B, preferably of influenza 2009 H1N1 and/or Influenza A subtypes H1 and/or H3 and/or Influenza B.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, as in the case of Influenza virus nucleic acids, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of about 80%, 85%, 90% or 95% homology or identity or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of one or more 2009 H1N1 influenza and/or influenza A and/or influenza B virus specific nucleic acids, preferably nucleic acids of Influenza A, e.g. of subtypes H1 and/or H3 and/or influenza 2009 H1N1 and/or Influenza B. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, throat or nasal swabs, saliva, and nasopharyngeal washes.

The term “patient” as used herein is meant to include both human and veterinary patients.

The amplification primers and detection probes of the present invention are set forth in the sequence listing.

In one aspect of the invention, there is provided a method for detection of 2009 H1N1 influenza and/or influenza A and/or influenza B in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detection of 2009 H1N1 influenza and/or influenza A and/or influenza B, preferably of Influenza A subtypes H1 and/or H3 and/or influenza 2009 H1N1 and/or of Influenza B in a sample comprising the steps of obtaining a sample from a patient; extracting nucleic acids from the sample; amplifying the nucleic acid, wherein the RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, throat or nasal swabs and nasopharyngeal washes.

In another embodiment of the invention, the assay is a component of a devices that is suitable in fully automated laboratories capable of extracting nucleic acids from a sample (e.g. using the epMotion System of Eppendorf International), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains.

All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXAMPLES

### Example 1

### Amplification of Influenza RNA

A real-time PCR with various primer combinations was performed, using RNA of Influenza A H1N1 (A/Virginia/ATCC3/2009; ATCC-Cat.-No. VR-1680TM), Influenza A/H3N2 (A/Aichi/2/68; ATCC-Cat.-No. VR-1738DTM), (2009H1N1), Influenza B RNA and control RNA as templates. Given names of primers and probes used herein as well as their sequences, their SEQ ID numbers, their classification as “Forward” or “Reverse” primers and probes, respectively, are depicted in Table A below. In said table, the key to degenerate nucleotides is as follows: R=A+G; M=A+C; W=A+T; K=G+T; S=G+C; Y=C+T; H=A+T+C; B=G+T+C; D=G+A+T; N=A+C+G+T; V=G+A+C
2009 H1N1 TaqMan probes are labeled with ROX reporter and BHQ-2 quencher;
Influenza B TaqMan probes are labeled with FAM reporter and BHQ-1 quencher;
Influenza A TaqMan probes are labeled with JOE reporter and BHQ-1 quencher.

TABLE A
Names, SEQ ID numbers, directions of primers and nucleotide sequences of
oligonucleotides according to the invention

| | | |
|---|---|---|
| H1N1 2009_HA_F011 (SEQ ID NO: 1) | Forward | GATGGATGGTACGGTTATCAC |
| H1N1 2009_HA_F012 (SEQ ID NO:2) | Forward | GATGGATGGTATGGTTATCACC |
| H1N1 2009_HA_R011 (SEQ ID NO: 3) | Reverse | CCTACTGCTGTGAACTGTG |
| RP02301ZXP011 (SEQ ID NO: 4) | Probe (2009H1N1) | AGCCGACCTGAAGAGCACAC |
| Influenza A_MP_F011 (SEQ ID NO: 5) | Forward | CGAGGTCGAAACGTATGTTC |
| Influenza A_MP_F012 (SEQ ID NO: 6) | Forward | CGAGGTTGACACGTACGTTC |
| Influenza A_MP_F013 (SEQ ID NO: 7) | Forward | AACCGAGGTCGAAACGTA |
| Influenza A_MP_R011 (SEQ ID NO: 8) | Reverse | CAGAGGTGACAAGATTGGTC |
| Influenza A_MP_R012 (SEQ ID NO: 9) | Reverse | CAGAGGTGACAGAATTGGTC |
| Influenza A_MP_R013 (SEQ ID NO: 10) | Reverse | AGAGGTGACAGGATTGGTC |
| RP02204ZXP011 (SEQ ID NO: 11) | Probe (Influ A) | GAGGCTCTCATGGAATGGCTAAAGACA |
| RP02204ZXP012 (SEQ ID NO: 12) | Probe (Influ A) | GAGGCTCTCATGGAGTGGCTAAAGACA |
| Influenza B_MP_F010 (SEQ ID NO: 13) | Forward | CAATTGCCTACCTGCTTTC |
| Influenza B_MP_R011 (SEQ ID NO: 14) | Reverse | GGTCAAACTCTTTCCCACC |
| Influenza B_MP_R012 (SEQ ID NO: 15) | Reverse | GGTCAAATTCTTTCCCACC |
| Influenza B_MP_R013 (SEQ ID NO: 16) | Reverse | AGTCAAATTCTTTCCCACC |
| Influenza B_MP_R014 (SEQ ID NO: 17) | Reverse | GATCAAATTCTTTCCCACC |
| RP02104ZXP010 (SEQ ID NO: 18) | Probe (Influ B) | CAGAAGATGGAGAAGGCAAAGCAGA |
| Influenza A_MP_F010 (SEQ ID NO: 19) | Forward | AACCGAGGTYGAMACGTA |
| Influenza A_MP_R010 (SEQ ID NO: 20) | Reverse | CAGAGGTGACARRATYGGTCT |
| Influenza A_MP_F010B (SEQ ID NO: 21) | Forward | CAGARACTKGAARRTGTCTTTGC |
| Influenza A_MP_R010B (SEQ ID NO: 22) | Reverse | ATYCCYTTAGTCAGAGGTGAC |
| Influenza B-mp F020 (SEQ ID NO: 23) | Forward | GACACAATTGCCTACCTGCTTTC |
| Influenza B-mp R021 (SEQ ID NO: 24) | Reverse | TCCCACCAAACCAACAGTGTAA |
| Influenza B-mp R022 (SEQ ID NO: 25) | Reverse | TCCCACCGAACCAACAGTGTAA |
| Influenza B-mp R023 (SEQ ID NO: 26) | Reverse | TCCCACCGAACCAACAATGTAA |

EC (Extraction control) primer and probe mixture were supplied by Bioline.

Primer sets listed in Table B were used in real time PCR reaction in this example.

TABLE B

| Primer | | | |
|---|---|---|---|
| combi- | Influenza A | Influenza B | 2009H1N1 |
| nation | template | template | template |
| Set A | F011, F012, F013, | F010, R012, R013, | F011, F012, R011 |
| | R011, R012, R013 | R014 | |
| Set B | F010, R010 | F010, R012, R013, | F011, F012, R011 |
| | | R014 | |
| Set C | F010B, R010B | F010, R012, R013, | F011, F012, R011 |
| | | R014 | |
| Set D | F011, F012, F013, | F020, R021, R022, | F011, F012, R011 |
| | R011, R012, R013 | R023 | |
| Set E | F010, R010 | F020, R021, R022, | F011, F012, R011 |
| | | R023 | |
| Set F | F010B, R010B | F020, R021, R022, | F011, F012, R011 |
| | | R023 | |

For Quanti-Tect mastermix program setting, RNA template was reverse transcribed to cDNA at 50° C. for 20 min. The RT enzyme was denatured at 94° C. for 15 minutes. Each PCR cycle was repeated 45 times at 94° C. for 45 s and 60° C. for 45 s. Each template was analyzed in triplicate and the CT value for each template and each set of primers was measured using the Rotor-gene Q real time cycler (Qiagen). The signal will be considered positive if the CT value is below 40. At the same time, a comparison between the performance of different sets of primers and probes can be determined by the CT value. The lower the CT value, the better the performance.

TABLE 1
QuantiTect (Qiagen)

| | Yellow | | Orange | | Green | | Red (Extraction | |
|---|---|---|---|---|---|---|---|---|
| | (influenza A) | | (2009H1N1) | | (influenza B) | | control) | |
| SetA | 17.76 | 18.01 | | | | | 25.63 | 26.01 |
| | 14.65 | 14.64 | | | | | 26.03 | 25.93 |
| | 21.38 | 21.14 | 21.76 | 21.58 | | | 25.57 | 25.88 |
| | | | | | 19.93 | 19.78 | 26.1 | 25.57 |
| | | | | | | | 25.54 | 25.29 |
| SetB | 17.83 | 17.99 | | | | | 26.02 | 26.28 |
| | 15.29 | 15.57 | | | | | 26.25 | 26.25 |
| | 22.53 | 22.01 | 21.74 | 21.55 | | | 25.65 | 25.39 |
| | | | | | 19.85 | 19.89 | 26.01 | 25.96 |
| | | | | | | | 25.99 | 25.49 |
| SetC | 18.59 | 18.84 | | | | | | |
| | 15.09 | 15.15 | | | | | | |
| | 22.69 | 22.59 | 21.6 | 21.64 | | | | |
| | | | | | 19.92 | 19.74 | | |
| SetD | 18.03 | 17.93 | | | | | 26.65 | 26.79 |
| | 14.61 | 14.74 | | | | | 27.29 | 26.92 |
| | 21.13 | 21.52 | 21.59 | 22.11 | | | 26.83 | 26.66 |
| | | | | | 20.45 | 20.26 | 27.39 | 26.72 |
| | | | | | | | 26.75 | 26.23 |
| SetE | 18.33 | 18.01 | | | | | 25.95 | 26.06 |
| | 15.46 | 15.68 | | | | | 26.11 | 26 |
| | 22.53 | 22.69 | 21.64 | 21.7 | | | 25.58 | 26.07 |
| | | | | | 20.38 | 20.35 | 25.7 | 25.62 |
| | | | | | | | 25.71 | 26 |
| SetF | 18.51 | 18.63 | | | | | 26.37 | 26.63 |
| | 14.98 | 15.02 | | | | | 26.1 | 26.06 |
| | 22.77 | 22.62 | 21.85 | 21.81 | 20.58 | 20.57 | 25.64 | 25.92 |
| | | | | | | | 25.84 | 26.24 |
| | | | | | | | 25.55 | 25.98 |

From the above results, it can be seen that for Influenza A template including H1N1, H3N2 and 2009 H1N1, the performance of the combination of primers and probes (e.g. in Set A) is better than that of primers with degenerate nucleotides (Sets E or F).

Advantageously and surprisingly the limit of detection (LOD) of 2009 H1N1 assay by multi-plex TaqMan Real-time PCR goes to as low as 1.5 copies of viral RNA per test. This has been determined in a test using serially diluted RNA. To this end, 10.8 μl of HawkZ05 Master mix (2.3×, Roche) and 1.75 μl of 25 mM of Mn(OAc)₂ were mixed.

5 μl of 20 μM primer mix containing Influenza A, B and H1N1 2009 primers were mixed together with 2.5 μl of 10 μM of influenza A, B and H1N1 2009 probes and added to the mixture. 20 μl of the final master mix was then aliquoted into individual PCR tubes. TMV pure RNA was serially diluted from 1 ng/μl to 10⁻⁷ with carrier RNA solution. Commercially synthesized positive control RNA for 2009 H1N1 from AmpTech which contains 2009 H1N1 forward primer and reverse primers and probes sequences was diluted in the same manner as TMV pure RNA. Equal amounts of the diluted RNA template were mixed together into the same tube before use. 5 μl of the template was later added to PCR tube that contains the mixture. The tube was then subjected to RT-PCR (RT at 55° C. for 5 minutes, 60° C. for 5 minutes, 65° C. for 5 minutes; followed by PCR reaction denaturation step at 94° C. for 5 second and annealing/extension step at 60° C. for 40 seconds.

In the above reaction, two templates were used, i.e. 2009 H1N1 control RNA and TMV RNA (control). Therefore, four sets of primer and probe mixture include Influenza A, Influenza B, 2009 H1N1 and TMV (primers and probes for the detection of TMV are found in the sequence listing).

The result showed that the LOD of the positive control RNA can go to 1.5 copies per test because in 5 μl of the diluted 0.1 ag positive control RNA which is around 1.5 copy per reaction the fluorescence is observed and CT value is less than 40.

The multiplex assay with four sets of primers and probes including the extraction control performs no much difference with the single-plex which only use one set of primer and probes for specific template. The mixing of 4 sets of primer and probes mix did not disturb the amplification of the template with its specific primer and probes.

### Assay Stability

The assay proved to be surprisingly robust. When different RT-PCR mastermixes were used (Quanti-fast (Qiagen), Quantitech (Qiagen) and Hawk Z05 (Roche) one step RT TaqMan Real-time PCR mastermix), the performance of the assays is quite similar. At the same time, the performance of the assay is quite similar over a broad range of annealing and extension temperatures from 53° C. to 65° C. when the CT values are compared.

The inventive Influenza A/B & 2009 H1N1 RT-PCR Test is a real-time PCR-based in vitro diagnostic test for the qualitative detection and differentiation of Influenza A, Influenza B and 2009 H1N1 (Pandemic Influenza A H1N1nv), e.g., in nasal swab and nasopharyngeal swab samples from human patients with signs and symptoms of respiratory tract infections. This diagnostic test kit is preferably for use with the Sentosa SX101 nucleic acid extraction platform, with the Sentosa SX Virus Total Nucleic Acid Kit, in conjunction with the Rotor-Gene Q MDX 5 plex HRM system.

Combined Kits and Devices

The combination of Sentosa SX101 and RGQ together with their softwares and in conjunction with the Sentosa SX Virus Total Nucleic Acid Kit and Sentosa SA Influenza A/B & 2009 H1N1 RT-PCR Test function as a whole sample extraction, PCR-setup and real-time PCR amplification and detection workflow for the verification and validation tests. The whole workflow takes less than 3 hours for 8 sample size.

### Sentosa SX101

The Sentosa SX101 is a flexible automated pipetting system that offers a unique, easy to use workflow for nucleic acid extraction and PCR setup for up to 4 assays simultaneously. With the Sentosa SX101 workflow, up to 48 samples can be processed simultaneously within 2 hours. The liquid (samples from the source tube) is transported in pipette tips and deposited in the destination tube. On request, an optical sensor automatically checks the correct selection and positioning of tubes, available supplies and the position of pipette tips in the rack, as well as liquid level in some tubes.

### Rotor-Gene Q MDX 5 Plex HRM

The RGQ instrument is designed to perform real-time thermal cycling, detection, and/or quantification using the polymerase chain reaction (PCR) in clinical applications. For detection, a set of 6 uncoupled excitation and emission filters are used in conjunction with 6 dedicated LEDs to maximize the detection capability. Each tube is illuminated by the excitation LED and the fluorescent signal transmitted to the PMT detector via a uniform optical path length. This eliminates well-to-well optical variation due to edge effect which block based cycler experience. It also allows for fluorescent detection without the need of a reference dye.

### Performance Tests—Analytical Sensitivity

The analytical limit of detection (LoD) was assessed for the Sentosa SA Influenza A/B & 2009 H1N1 RT-PCR Test using four strains of Influenza extracted on the Sentosa SX101 instrument. Initial 10-fold serial dilutions were performed. The LoD was determined for 2009 H1N1, Influenza A and Influenza B using A/Virginia/ATCC2/2009 (H1N1) (VR-1737), A/Victoria/3/75 (H3N2) (VR-822), B/Allen/45 (VR-102), A/NWS/33 (H1N1) (VR-219). An initial estimate of LoD was obtained using serial dilutions of viruses tested in triplicates for each dilution. The limit of detection was confirmed by extraction and amplification of 23 replicates. The LoD was determined to be the dilution at which at least 95% of the replicates were positive. The unit (copies/uL) of the virus is quantified by standard curve from serial dilution of PC RNA.

The tested strains were quantified by the established standard curve by the positive control RNA in each target channel and the LoD is claimed based on highest virus titer tested which gives more than 95% total agreement and less than 100% extraction efficiency.

TABLE 2
Test results

| | | | | Copy |
|---|---|---|---|---|
| Strain | Subtype | LoD | | number/uL |
| 2009 H1N1 | A | 10²-10³ | TCID₅₀/mL | 4 |
| Influenza A/ | A | 10⁰-2 × 10⁰ | CEID₅₀/mL | 1 |
| H3N2 | | | | |
| Influenza A/ | A | 10⁰-2 × 10⁰ | CEID₅₀/mL | 1 |
| H1N1 | | | | |
| Influenza B | B | 2.5 × 10⁰ | CEID₅₀/mL | 3 |

### Analytical Reactivity and Specificity

Both reactivity and specificity tests include virus extraction and real-time PCR amplification to demonstrate that (1) the assay is designed to detect all strains of Influenza tested; and (2) there is no cross-reactivity with other clinically isolated microorganisms tested. A variety of viruses as well as bacterial strains obtained from the ATCC were used to test the specificity of the assay.

Test Result

The tested strains were quantified by the established standard curve by the positive control RNA in each target channel and the LoD is claimed based on highest virus titer tested which gives more than 95% total agreement and less than 100% extraction efficiency.

### Tables 3A and 3B—Influenza Detection Sensitivity and Specificity

| | | | | | Extraction |
|---|---|---|---|---|---|
| | | Influenza Detection | | | Control |
| | | Green | Yellow | Orange | Red |
| Virus, Strain | Source | Channel | Channel | Channel | channel |
| A/Swine/1976/31 (H1N1) | ATCC | − | + | − | + |
| A/Swine/Iowa/15/30 (H1N1) | ATCC | − | + | − | + |
| A/Aichi/2/68 (H3N2) | ATCC | − | + | − | + |
| Novel influenza 1 H1N1 RNA | Vircell | + | + | − | + |
| control, A/California/07/2009 | | | | | |
| (H1N1) | | | | | |
| Influenza A H1 RNA control, | Vircell | − | + | − | + |
| A/Brisbane/59/2007(H1N1) | | | | | |
| Influenza A H3 RNA control, | Vircell | − | + | − | + |
| A/Perth/16/2009(H3N2) -H3 | | | | | |
| Influenza A H5 RNA control, | Vircell | − | + | − | + |
| A/reassortant/NIBRG-14 | | | | | |
| (Vietnam/1194/2004 × Puerto | | | | | |
| Rico/8/1934) (H5N1) | | | | | |
| Influenza B RNA control, | Vircell | − | − | + | + |
| B/Brisbane/60/2008 | | | | | |
| A/Virginia/ATCC1/2009 (H1N1) | ATCC | + | + | − | + |
| A/FM/1/47 (H1N1) | ATCC | − | + | − | + |
| A/Weiss/43 (H1N1) | ATCC | − | + | − | + |
| A/Port Chalmers/1/73 (H3N2) | ATCC | − | + | − | + |
| B/GL/1739/54 | ATCC | − | − | + | + |
| B/Hong Kong/5/75 | ATCC | − | − | + | + |
| A/Christ Church/1/2003 (H1N1) | NIBSC | − | + | − | + |
| A/Wyoming/3/2003 (H3N2) | NIBSC | − | + | − | + |
| B/Jiangsu/10/2003 | NIBSC | − | − | + | + |
| A/NWS/33 (H1N1) | ATCC | − | + | − | + |
| A/New Jersey/8/76 (H1N1) | ATCC | − | + | − | + |
| B/Maryland/1/59 | ATCC | − | − | + | + |
| B/Taiwan/2/62 | ATCC | − | − | + | + |
| A/Denver/1/57 (H1N1) | ATCC | − | + | − | + |
| A/Aichi/2/68 (H3N2) | ATCC | − | + | − | + |
| A/Victoria/3/75 (H3N2) | ATCC | − | + | − | + |
| B virus, B/Lee/40 | ATCC | − | − | + | + |
| B/Allen/45 | ATCC | − | − | + | + |
| A/Virginia/ATCC2/2009 | ATCC | + | + | − | + |
| A/Virginia/ATCC3/2009 | ATCC | + | − | − | + |
| A/Hong Kong/8/68 (H3N2) | ATCC | − | + | − | + |
| A/PR/8/34 (H1N1) | ATCC | − | + | − | + |

TABLE 3B

| | | | | Extraction |
|---|---|---|---|---|
| | Influenza Detection | | | Control |
| | Green | Yellow | Orange | Red |
| Organism, Strain | channel | channel | channel | channel |
| Coronavirus (Human), strain 229E | − | − | − | + |
| Coronavirus (Human), strain OC43 | − | − | − | + |
| Enterovirus type 71, BrCt | − | − | − | + |
| Human Parainfluenza virus 1, strain C-35 | − | − | − | + |
| Human Parainfluenza virus 2, Greer | − | − | − | + |
| Human Parainfluenza virus 3, C243 | − | − | − | + |
| Human Parainfluenza virus 4a | − | − | − | + |
| Human Parainfluenza virus 4b, CH 19503 | − | − | − | + |
| Human Metapneumovirus | − | − | − | + |
| Human Respiratory Syncytial virus (B1 wt), | − | − | − | + |
| BWV/14617/85 | | | | |
| Human Respiratory Syncytical virus, strain A2 | − | − | − | + |
| Human Respiratory Syncytical virus, strain 9320 | − | − | − | + |
| Human Respiratory Syncytical virus, Strain Long | − | − | − | + |
| Human Respiratory Syncytical virus, strain 18537 | − | − | − | + |
| Measles virus, Edmonston | − | − | − | + |
| Mumps, Jones | − | − | − | + |
| Rhinovirus 87, strain FO2-3607 Com | − | − | − | + |
| Rhinovirus type 2, strain HGP | − | − | − | + |
| Rhinovirus 57, strain Ch47 | − | − | − | + |
| Adenovirus Type 31, strain 1315/63 | − | − | − | + |
| Adenovirus type 35, strain Holden | − | − | − | + |
| Adenovirus type 14, strain de Wit | − | − | − | + |
| Adenovirus type 11, strain Slobitski | − | − | − | + |
| Adenovirus Type 7, strain Gomen | − | − | − | + |
| Adenovirus 1, Strain Adenoid 71 | − | − | − | + |
| Bordetella Pertussis gDNA | − | − | − | + |
| Chlamydophila puemoniae, strain TW-183 | − | − | − | + |
| Corynebacterium diphtheriae, NCTC 13129 | − | − | − | + |
| Cytomegalovirus (huHerpesvirus 5), strain Davis | − | − | − | + |
| Coccidioidies Immitis DNA control, 50 ng/ul | − | − | − | + |
| Epstein Barr virus, strain P-3 | − | − | − | + |
| Escherichia Coli O103, strain NCDC H515b | − | − | − | + |
| Haemophilus influenza, ATCC 51907 | − | − | − | + |
| Klebsiella pneumonia gDNA, strain ART2008133 | − | − | − | + |
| Lactobacillus acidophilus, ATCC 314 | − | − | − | + |
| Legionella pneumophila, strain Philadelphia-1 | − | − | − | + |
| Moraxella catarrhalis, strain 20 | − | − | − | + |
| Mycoplasma pneumonia, ATCC 29342 | − | − | − | + |
| Mycobacterium tuberculosis, strain H37Ra | − | − | − | + |
| Mycobacterium tuberculosis, strain H37Rv | − | − | − | + |
| Mycobacterium intracellulare DNA control, quantified | − | − | − | + |
| Neisseria Meningitidis gDNA serotype A | − | − | − | + |
| Neisseria Meningitidis gDNA serotype B | − | − | − | + |
| Neisseria Meningitidis gDNA serotype C | − | − | − | + |
| Neiseeria Sicca | − | − | − | + |
| Neisseria Meningitidis gDNA, strain M1883 | − | − | − | + |
| Pseudomonas aeruginosa gDNA, strain Boston 41501 | − | − | − | + |
| Pseudomonas aeruginosa, strain 16 | − | − | − | + |
| Neisseria sp - Neisseria Subflava | − | − | − | + |
| Staphylococcus epidermidis, FDA strain PCI 1200 | − | − | − | + |
| Staphylococcus aureus, strain TCH1516 | − | − | − | + |
| Staphylococcus aureus, strain FPR3757 | − | − | − | + |
| Staphylococcus epidermis gDNA | − | − | − | + |
| Streptococcus pneumonia, CIP 104225 | − | − | − | + |
| Streptococcus pyogenes, M-type 66 | − | − | − | + |
| Streptococcus pyogenes, MGAS9429 serotype M12 | − | − | − | + |
| Streptococcus pyogenes, SF370 | − | − | − | + |
| Streptococcus Pyogenes RosenBach | − | − | − | + |
| Streptococcus salivarius, strain DSM 13084 | − | − | − | + |
| Streptococcus pneumoniae | − | − | − | + |

Further, A panel of Influenza samples (Influenza virus A and B, 2011 EQA Program) from Quality Control for Molecular Diagnostics (QCMD), which is an independent International External Quality Assessment (EQA) organization, was tested using the inventive Influenza A/B & 2009 H1N1 RT-PCR Test.

| Sample | Sample Content | Sample Status | Results |
|---|---|---|---|
| INFRNA 11-05 | Influenza Virus H1N1 | Frequently | Detected |
| | PDM09 | detected | |
| INFRNA 11-01 | Influenza Virus H1N1 | Frequently | Detected |
| | PDM09 | detected | |
| INFRNA 11-08 | Influenza Virus H1N1 | Detected | Not detected |
| | PDM09 | | |
| INFRNA 11-11 | Influenza Virus H3N2 | Frequently | Detected |
| | | detected | |
| INFRNA 11-10 | Influenza Virus H3N2 | Detected | Detected |
| INFRNA 11-02 | Influenza Virus H3N2 | Infrequently | Not detected |
| | | detected | |
| INFRNA 11-09 | Influenza Virus B | Detected | Detected |
| | (Victoria) | | |
| INFRNA 11-04 | Influenza Virus B | Infrequently | Not detected |
| | (Victoria) | detected | |
| INFRNA 11-12 | Influenza Virus B | Frequently | Detected |
| | (Yamagata) | detected | |
| INFRNA 11-07 | Influenza Virus B | Detected | Detected |
| | (Yamagata) | | |
| INFRNA 11-03 | Influenza A & B | Negative | Not detected |
| | Negative | | |
| INFRNA 11-06 | Influenza A & B | Negative | Not detected |
| | Negative | | |

184 nasal swab specimens that have been tested by D3 Ultra DFA Respiratory Virus Screening assessed clinical performance characteristics of the inventiveInfluenza A/B & 2009 H1N1 RT-PCR Test and ID Kit combined with virus culture.

### Test Results

The clinical sensitivity and specificity for this assay is calculated as below:

TABLE 4

| | | Test results by reference methods | | |
|---|---|---|---|---|
| | | Negative | Positive | Total |
| Tested results by | Negative | 132 | 1 | 133 |
| inventive Influenza | Positive | 2 | 49 | 51 |
| A/B & 2009 H1N1 | All | 134 | 50 | 184 |
| RT-PCR Test | | | | |

Calculated clinical sensitivity and specificity

TABLE 5
Performance Measures

| | | Lower | Upper |
|---|---|---|---|
| Measure | Estimate | 95% CI | 95% CI |
| Prevalence | 27.2% | 21.3% | 34.0% |
| Clinical sensitivity | 98% | 89.5% | 99.6% |
| Clinical specificity | 98.5% | 94.7% | 99.6% |

The clinical performance of this whole workflow is competitive or even better than the Pro Flu+ and Pro Flu fast from Gen-Probe, artus Infl A/B RG RT-PCR Kit, Xpert Flu Assay from Cepheid.

### Reproducibility

Reproducibility data permit a regular performance assessment of the inventive Influenza A/B & 2009 H1N1 RT-PCR Test as well as an efficiency comparison with other products. The inter/intra-assay reproducibility was determined by testing 3 concentrations of RNA in triplicates together with a negative control (RNA NC), a positive control (Influenza PC) and water (NTC). The overall reproducibility assessment setup allows to test intra-assay variability (variability of multiple results of samples of the same concentration within one experiment), the inter-assay variability (variability of multiple results of the assay generated on different instruments of the same type by different operators within one laboratory) and the inter-batch variability (variability of multiple results of the assay using various batches of mastermix and primer/probe mix).

TABLE 6
Test results

| | Agreement | | | | | |
|---|---|---|---|---|---|---|
| | with | | | | | |
| | expected | | Total Agreement with | | | |
| Channels | results | Total runs | expected results (%) | | 95% Confidence Interval* | |
| Green | 260 | 260 | 260/260 | 100.00% | 98.54% | 100.00% |
| Yellow | 259 | 260 | 259/260 | 99.62% | 98.17% | 99.62% |
| Orange | 260 | 260 | 260/260 | 100.00% | 98.54% | 100.00% |
| Red | 258 | 260 | 258/260 | 99.23% | 97.79% | 99.24% |
| Total Agreement | 1037 | 1040 | 1037/1040 | 99.71% | 99.34% | 99.71% |
| All | | | | | | |

*Wilson (Score) 95% Confidence Interval

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the simultaneous detection of the presence or absence of at least one nucleic acid of 2009 H1N1 influenza and/or influenza A and/or influenza B in a biological sample, wherein the method comprises:
(a) isolating nucleic acids from the biological sample and optionally performing a reverse transcription step, and
(b) conducting real-time PCR, wherein primer sets are specific for 2009 H1N1 influenza and/or influenza A and/or influenza B,
wherein the 2009 H1N1 influenza-specific primer set comprises oligonucleotides selected from sequences set forth in SEQ ID NOs: 1 to 3, or complements or sequences having at least about 80%, 85%, 90% or 95% homology or identity, and
wherein the influenza A-specific primer set comprises oligonucleotides selected from sequences set forth in SEQ ID NOs: 5 to 10 and 19 to 22, or complements or sequences having at least about 80%, 85%, 90% or 95% homology or identity, and
wherein the influenza B-specific primer set comprises oligonucleotides selected from sequences set forth in SEQ ID NOs: 12 to 16 and 23 to 26, or complements or sequences having at least about 80%, 85%, 90% or 95% homology or identity, and
wherein at least one probe specifically binding to a nucleic acid of 2009 H1N1 influenza and/or at least one probe specifically binding to a nucleic acid of influenza A and/or at least one probe specifically binding to a nucleic acid of influenza B are used, wherein at least one probe or two, three or four probes are selected from the oligonucleotides set forth in SEQ ID NOs: 4, 11, 12 and 18, complements or probes having 80%, 85%, 90% or 95% homology or identity to the oligonucleotides set forth in SEQ ID NOs: 4, 11, 12 and 18 or their complements.

2. The method according to claim 1, wherein the primers and/or probes carry a fluorescent moiety.

3. An in vitro method for the diagnosis of an influenza virus infection in a subject comprising performing the method according to claim 1.

4. A method for monitoring the treatment of influenza virus infection, said method comprising performing the method according to claim 3 before treatment with at least one anti-viral drug and during and/or after treatment with said anti-viral drug.

5. A real-time PCR assay for simultaneous detection of at least one nucleic acid of 2009 H1N1 influenza and/or influenza A and/or influenza B in a biological sample comprising primers specifically hybridizing to nucleic acids derived from said 2009 H1N1 influenza and/or influenza A and/or influenza B, having oligonucleotide sequences as defined in claim 1.

6. The assay according to claim 5, wherein the assay is adapted for use in a fully automated laboratory.

7. A composition comprising primers and/or probes having oligonucleotide sequences as set forth in claim 1.

8. A kit for the simultaneous detection of 2009 H1N1 influenza and/or influenza A and/or influenza B in a biological sample comprising primers and/or probes having oligonucleotide sequences as set forth in claim 1 and instructions for use.

9. The kit according to claim 8, wherein said kit further comprises enzymes, deoxynucleotides, and/or buffers for performing a reverse transcription step and/or a PCR step.

10. The kit according to claim 8, further comprising reagents for the isolation of nucleic acids from a biological sample.


SEQUENCE LISTING (29 sequences)

SEQ ID NO: 1 - DNA, 21 bp, Influenza A virus
gatggatggtacggttatcac

SEQ ID NO: 2 - DNA, 22 bp, Influenza A virus
gatggatggtatggttatcacc

SEQ ID NO: 3 - DNA, 19 bp, Influenza A virus
cctactgctgtgaactgtg

SEQ ID NO: 4 - DNA, 20 bp, Influenza A virus
agccgacctgaagagcacac

SEQ ID NO: 5 - DNA, 20 bp, Influenza A virus
cgaggtcgaaacgtatgttc

SEQ ID NO: 6 - DNA, 20 bp, Influenza A virus
cgaggttgacacgtacgttc

SEQ ID NO: 7 - DNA, 18 bp, Influenza A virus
aaccgaggtcgaaacgta

SEQ ID NO: 8 - DNA, 20 bp, Influenza A virus
cagaggtgacaagattggtc

SEQ ID NO: 9 - DNA, 20 bp, Influenza A virus
cagaggtgacagaattggtc

SEQ ID NO: 10 - DNA, 19 bp, Influenza A virus
agaggtgacaggattggtc

SEQ ID NO: 11 - DNA, 27 bp, Influenza A virus
gaggctctcatggaatggctaaagaca

SEQ ID NO: 12 - DNA, 27 bp, Influenza A virus
gaggctctcatggagtggctaaagaca

SEQ ID NO: 13 - DNA, 19 bp, Influenza B virus
caattgcctacctgctttc

SEQ ID NO: 14 - DNA, 19 bp, Influenza B virus
ggtcaaactctttcccacc

SEQ ID NO: 15 - DNA, 19 bp, Influenza B virus
ggtcaaattctttcccacc

SEQ ID NO: 16 - DNA, 19 bp, Influenza B virus
agtcaaattctttcccacc

SEQ ID NO: 17 - DNA, 19 bp, Influenza B virus
gatcaaattctttcccacc

SEQ ID NO: 18 - DNA, 25 bp, Influenza B virus
cagaagatggagaaggcaaagcaga

SEQ ID NO: 19 - DNA, 18 bp, Influenza A virus
aaccgaggtygamacgta

SEQ ID NO: 20 - DNA, 21 bp, Influenza A virus
cagaggtgacarratyggtct

SEQ ID NO: 21 - DNA, 23 bp, Influenza A virus
cagaractkgaarrtgtctttgc

SEQ ID NO: 22 - DNA, 21 bp, Influenza A virus
atyccyttagtcagaggtgac

SEQ ID NO: 23 - DNA, 23 bp, Influenza B virus
gacacaattgcctacctgctttc

SEQ ID NO: 24 - DNA, 22 bp, Influenza B virus
tcccaccaaaccaacagtgtaa

SEQ ID NO: 25 - DNA, 22 bp, Influenza B virus
tcccaccgaaccaacagtgtaa

SEQ ID NO: 26 - DNA, 22 bp, Influenza B virus
tcccaccgaaccaacaatgtaa

SEQ ID NO: 27 - DNA, 18 bp, Tobacco mosaic virus
gacgtgcgaattcctcag

SEQ ID NO: 28 - DNA, 21 bp, Tobacco mosaic virus
gttgatgttgcatactggttg

SEQ ID NO: 29 - DNA, 25 bp, Tobacco mosaic virus
aacctcttgaactgacagcgtgtgc